(19) United States
(12) Patent Application Publication
(10) Pub. No.: US 2008/0027442 A1
(43) Pub. Date: Jan. 31, 2008

(54) Cranial fixation device and method for fixing same

(76) Inventor: James Blue, Mercer Island, WA (US)

Correspondence Address: OLYMPIC PATENT WORKS PLLC, P.O. BOX 4277, SEATTLE, WA, 98104, US

(21) Appl. No.: 11/827,788

(22) Filed: Jul. 13, 2007

Related U.S. Application Data
(60) Provisional application No. 60/875,123, filed on Dec. 15, 2006.
(60) Provisional application No. 60/833,317, filed on Jul. 25, 2006.

(51) Int. Cl. A61B 17/56

(52) U.S. Cl. 606/72

(57) ABSTRACT

Various embodiments of the present invention are directed to a cranial fixation device for fixing a skull flap to a skull. The cranial fixation device may be securely fastened inside a craniotomy-created perimeter burr hole so that a top surface of the cranial fixation device is flush with an outer surface of the skull and the skull flap. In one embodiment of the present invention, the cranial fixation device includes a base, a stem, and a fan washer. The base may be positioned beneath the burr hole with the interconnected stem projecting through the burr hole. The fan washer includes a fan-washer interconnection aperture and a number of peripheral fan blades. The fan washer interconnects to the stem via the fan-washer interconnection aperture and may be slid down the stem and held at a desired position within the burr hole by the peripheral fan blades.

### CROSS-REFERENCE TO RELATED APPLICATION

This application claims the benefit of Provisional Application No. 60/833,317, filed Jul. 25, 2006 and Provisional Application No. 60/875,123, filed Dec. 15, 2006.

### TECHNICAL FIELD

The present invention relates to the field of neurosurgical instrumentation, and, in particular, to a cranial fixation device.

### BACKGROUND OF THE INVENTION

A craniotomy is a surgical procedure performed by neurosurgeons to obtain access to the brain and other intracranial structures. Craniotomies may be performed for many reasons, including the removal of tumors, traumatic hematomas, spontaneous hematomas, infections, epileptic foci, and abscesses, as well as for the correction of cerebral aneurysms and other situations where access to the brain and/or intracranial structures is necessary. Typically, a surgeon entering a patient's skull cuts a flap of skin around the region of the skull to be opened and retracts the flap of skin to expose the outer surface of the skull. FIG. 1 shows the retraction of a skin flap away from the skull of a patient. Once the skin is peeled away from the site of the craniotomy, a surgeon drills a number of burr holes at locations around the perimeter of the area of the brain to be exposed, using a cranial perforator, or other suitable drilling device. FIG. 2 shows a typical 14 mm cranial perforator for drilling burr holes in a patient's skull. FIG. 3 shows a cranial perforator drilling a burr hole in a patient's skull. FIG. 4 shows three burr holes drilled into the skull of a patient.

Once the burr holes are drilled, the dura mater (“dura”), a tough fibrous membrane covering the brain, needs to be stripped away from the undersurface of the skull between adjacent burr holes. Stripping the dura away from the underside of the skull minimizes damage to the dura and the brain underneath when a portion of the skull is subsequently cut and removed. Stripping the dura from the underside of the skull is often performed by carefully inserting a curved blunt dissector through each of the burr holes and between the dura and an inner surface of the skull, and using the curved blunt dissector to gently strip the dura from the skull. It is desirable to make the span between any two adjacent burr holes less than twice the reach of the curved blunt dissector, so that the curved blunt dissector may be inserted into any two adjacent burr holes and completely strip the dura between the burr holes.

Once the dura is stripped away, a surgeon may use a side-cutting saw, or craniotome, to create a saw-cut line through the skull between adjacent burr holes to produce a removable piece of skull called a “skull flap.” A skull flap may either be completely removed from the patient, or the skull flap may be left attached to the skin and muscles of the flap of cut skin. Once the skull flap is created and removed from the skull, the exposed dura may then be opened under direct visualization, thus reducing the risk of injury to the underlying brain tissue.

At the end of the surgical procedure, the skull flap is returned to its former position and fixed in place. Typically, fixation is accomplished by drilling small, roughly 1-2 mm diameter, holes along the edge of the skull flap that align with similarly drilled holes located along the skull surrounding the skull flap. FIG. 5 shows a small hole being drilled into the skull surrounding the skull flap of a patient. The skull flap 502 includes a number of small skull-flap fixation apertures drilled through the skull flap 502, such as skull-flap fixation aperture 504. The region of the patient's skull surrounding the skull flap 502 contains a number of small perimeter fixation apertures, such as perimeter fixation aperture 506, which are positioned adjacent to each skull-flap fixation aperture when the skull flap 502 is fixed in place. In FIG. 5, wire, or suture 508, is shown passing through two of the perimeter fixation apertures, including perimeter fixation aperture 506. When the skull flap 502 is replaced in its former position, the wire 508 may additionally be passed through the skull-flap fixation aperture 504 and tied together to secure the skull flap 502 in place. This process may be repeated for each adjacent skull-flap fixation aperture and perimeter fixation aperture. The skull flap 502 shown in FIG. 5 includes partial burr holes 510-513 at each of the four corners of the skull flap 502. The partial burr holes 510-513 were formed during removal of the skull flap 502. When the skull flap 502 is fixed in place, the partial burr holes 510-513 align with partial burr holes 516-519, respectively, at the corners of the cut skull to re-create complete burr holes. Typically, during fixation, the burr holes are either left open or filled with bone chips produced during the drilling of the burr holes.

Although the fixation technique described above, with reference to FIG. 5, is adequate for brain protection, this fixation technique often results in undesirable cosmetic consequences, including a depression in the skin covering a sunken skull flap or one or more indentations in the skin covering the burr holes. Various alternate fixation techniques have recently been developed to improve post-operative cosmetics. A first alternate fixation technique utilizes small plates, bars, and/or rivet-like devices that may be fastened to an outer surface of a skull and an outer surface of an adjacent skull flap along saw-cut lines to fix the skull flap to the skull and prevent subsequent sinking of the skull flap. FIG. 6 shows a metal plate with a removable placement handle and a metal screw that may be used for fixing a skull flap to a skull. FIGS. 7 and 8 each show a metal bar screwed to a number of pieces of skull that fix the skull pieces together. FIG. 9 shows a cross-sectional view of a skull with a metal plate and two metal bars that are being used to fix the skull flap to the skull. FIG. 10 shows a skull with a skull flap that has been fixed to the skull by placing a rivet-like device along each saw-cut line between burr holes. A second alternate fixation technique utilizes large (12-16 mm diameter) plastic and/or metal rivet-like devices placed over top of burr holes and screwed to the outer surfaces of both the skull and the skull flap adjacent to the burr holes.

Neither of the two alternate fixation techniques eliminates cosmetic problems associated with a craniotomy. Although the sinking of the skull flap and/or the open burr holes may be ameliorated using one, or both, of the alternative fixation techniques, the plates, bars, and rivets used in each alternate fixation technique are placed on top of the outer surface of the skull and skull flap. Consequently, the plates, bars, and/or rivet-like devices create a palpable and often visible, permanent post-operative bump beneath the skin. This is particularly problematic where the scalp is normally thinner, such as in the forehead and facial region and in patients with generally thin scalps.

In the case of the rivet-like devices being placed along the saw-cut lines, the dura needs to be stripped further away from the edge of the skull opening in order to seat a base plate beneath the skull. This often causes bleeding in the epidural space that may be difficult to control, but needs to be addressed prior to closure. Additionally, when rivet-like devices or plates are being placed over the burr holes to increase cosmesis, many surgeons try to perform their craniotomies with the fewest possible burr holes and to avoid drilling burr holes altogether in areas that will be visible after the surgery, such as the forehead. This reluctance to generously place burr holes around the perimeter of a planned exposure site may increase the risk of dural injury, and, more importantly, brain injury when using the craniotome. Neurosurgeons, health-care providers, and patients undergoing craniotomies have, therefore, recognized a need for an improved way to fix skull flaps to skulls that increases post-operative cosmesis.

### SUMMARY OF THE INVENTION

Various embodiments of the present invention are directed to a cranial fixation device for fixing a skull flap to a skull. The cranial fixation device may be securely fastened inside a craniotomy-created perimeter burr hole so that a top surface of the cranial fixation device is flush with an outer surface of the skull and the skull flap. In one embodiment of the present invention, the cranial fixation device includes a base, a stem, and a fan washer. The base may be positioned beneath the burr hole with the interconnected stem projecting through the burr hole. The fan washer includes a fan-washer interconnection aperture and a number of peripheral fan blades. The fan washer interconnects to the stem via the fan-washer interconnection aperture and may be slid down the stem and held at a desired position within the burr hole by the peripheral fan blades.

### BRIEF DESCRIPTION OF THE DRAWINGS

FIG. 1 shows the retraction of a skin flap away from the skull of a patient.

FIG. 2 shows a typical 14 mm cranial perforator for drilling burr holes in a patient's skull.

FIG. 3 shows a cranial perforator drilling a burr hole in a patient's skull.

FIG. 4 shows three burr holes drilled into the skull of a patient.

FIG. 5 shows a small hole being drilled into the skull surrounding the skull flap of a patient.

FIG. 6 shows a metal plate with a removable placement handle and a metal screw that may be used for fixing a skull flap to a skull.

FIGS. 7 and 8 each show a metal bar screwed to a number of pieces of skull that fix the skull pieces together.

FIG. 9 shows a cross-sectional view of a skull with a metal plate and two metal bars that are being used to fix the skull flap to the skull.

FIG. 10 shows a skull with a skull flap that has been fixed to the skull by placing a rivet along each saw-cut line between burr holes.

FIG. 11A shows three orthogonal views of a base and stem for a fixation device that represents one embodiment of the present invention.

FIGS. 11B and 11C show a top view and a cross-sectional view of a fan washer for a fixation device that represents one embodiment of the present invention.

FIGS. 11D and 11E show a top view and a cross-sectional view of a cap for a fixation device that represents one embodiment of the present invention.

FIG. 11F shows three side views of a fixation device that represents one embodiment of the present invention.

FIG. 12 shows a cross-sectional view of a burr hole drilled into a skull by a drill bit from a cranial perforator.

FIG. 13A shows a first step for fixing a skull flap to a skull using the fixation device shown in FIGS. 11A-11F that represents one embodiment of the present invention.

FIG. 13B shows a second step for fixing a skull flap to a skull using the fixation device shown in FIGS. 11A-11F that represents one embodiment of the present invention.

FIG. 13C shows a third step for fixing a skull flap to a skull using the fixation device shown in FIGS. 11A-11F that represents one embodiment of the present invention.

FIG. 13D shows a fourth step for fixing a skull flap to a skull using the fixation device shown in FIGS. 11A-11F that represents one embodiment of the present invention.

FIG. 14 shows a skull flap fixed to a skull by four fixation devices that represent one embodiment of the present invention.

FIG. 15A shows a side view of a base and an interconnected stem for a fixation device that represents one embodiment of the present invention.

FIG. 15B shows a top view of a base and an interconnected stem for a fixation device that represents one embodiment of the present invention.

FIG. 15C shows a top view of a cap for a fixation device that represents one embodiment of the present invention.

FIG. 15D shows a cross-sectional view of a fan washer for a fixation device that represents one embodiment of the present invention.

FIG. 16A shows a first step for fixing a skull flap to a skull using the fixation device shown in FIGS. 15A-15D that represents one embodiment of the present invention.

FIG. 16B shows a second step for fixing a skull flap to a skull using the fixation device shown in FIGS. 15A-15D that represents one embodiment of the present invention.

FIG. 16C shows a third step for fixing a skull flap to a skull using the fixation device shown in FIGS. 15A-15D that represents one embodiment of the present invention.

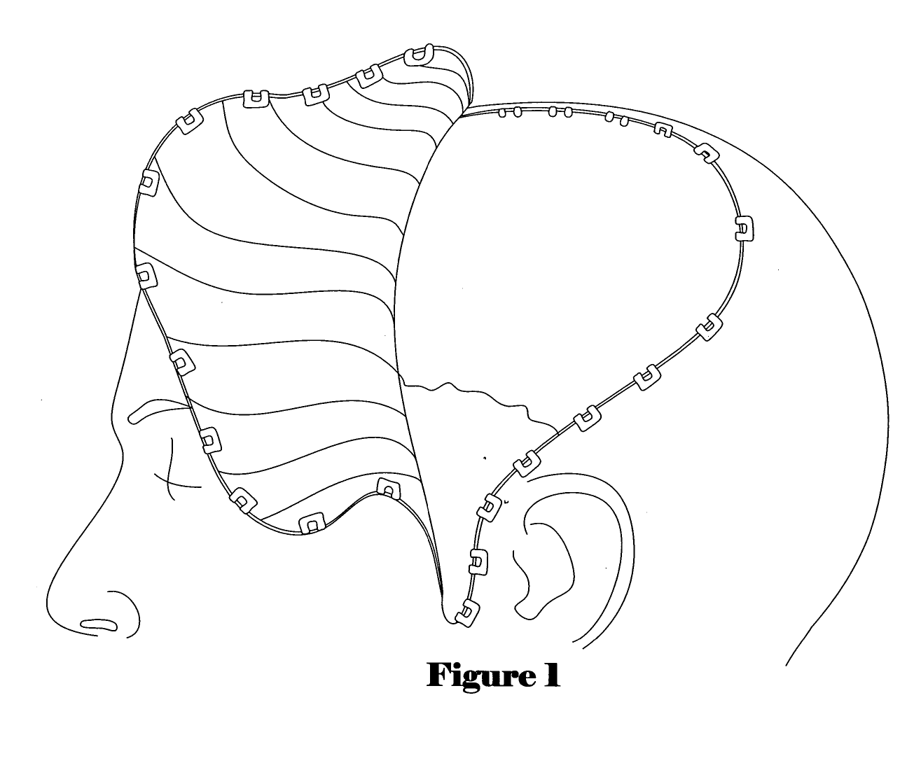

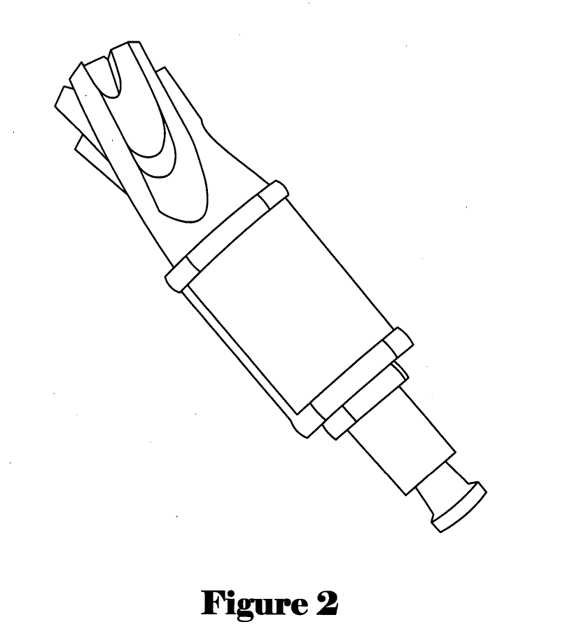

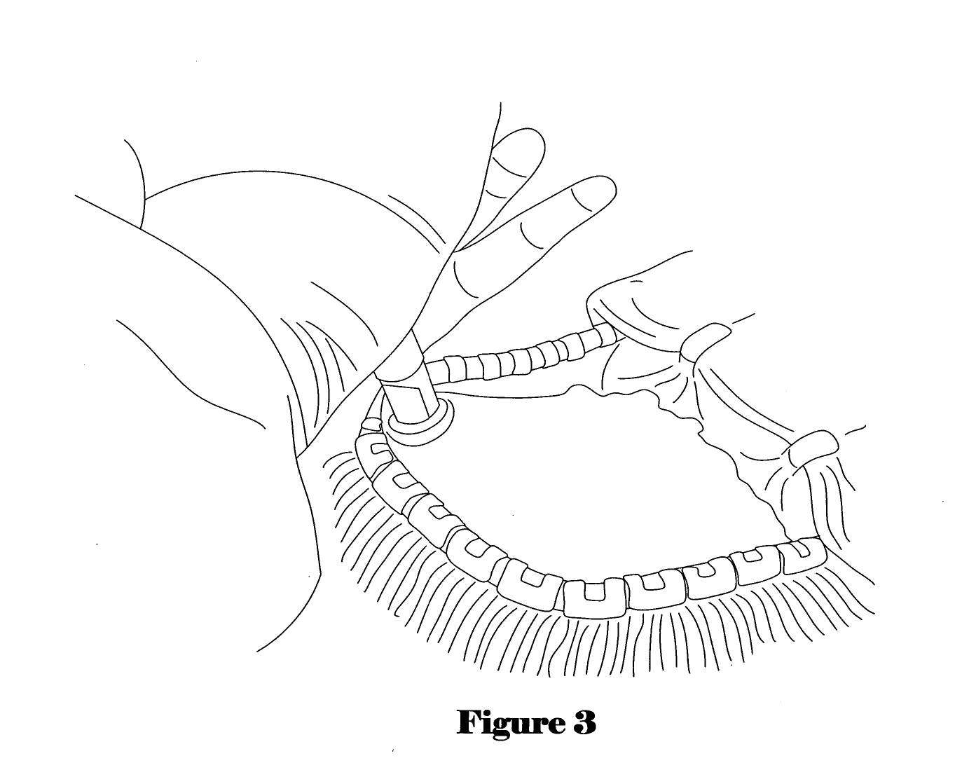

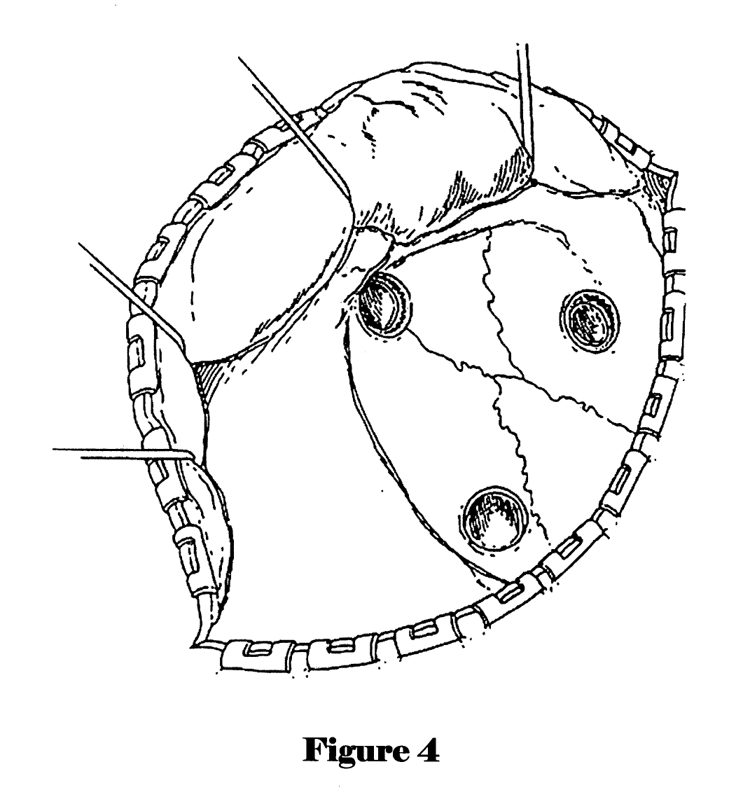

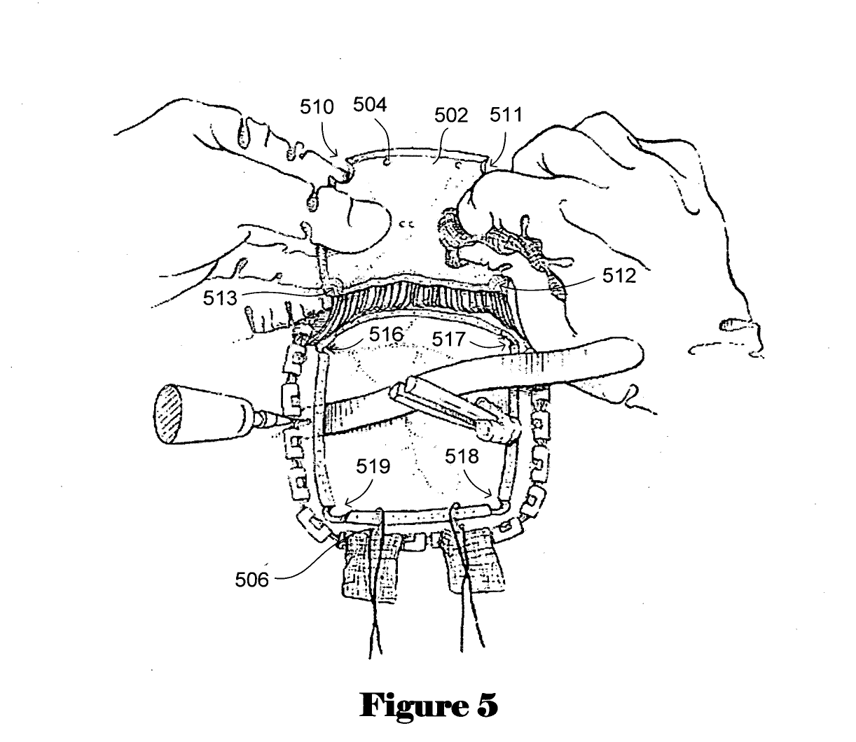

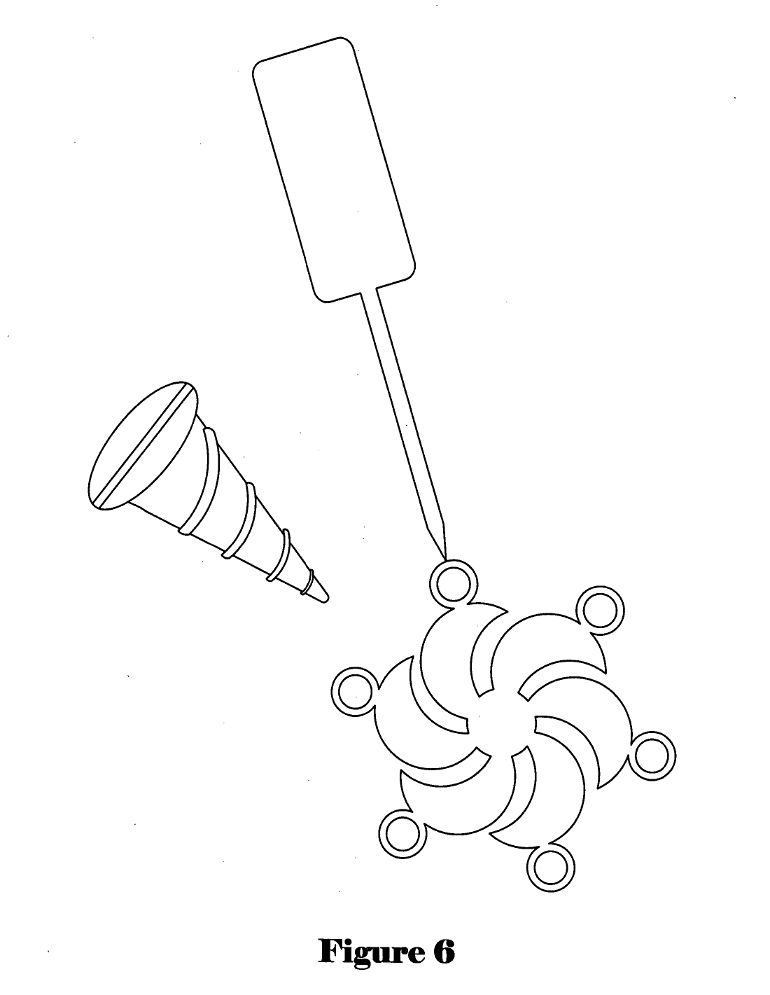

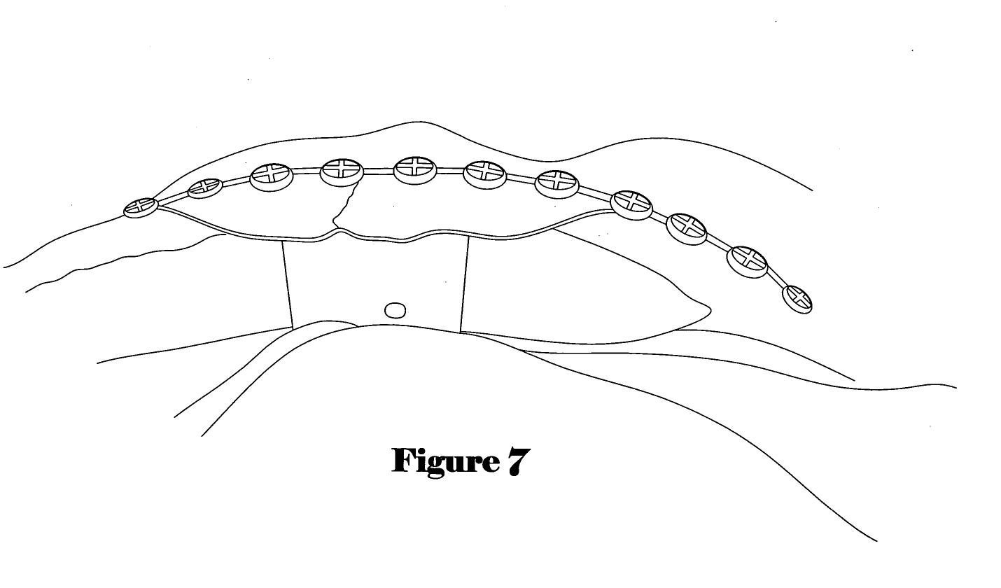

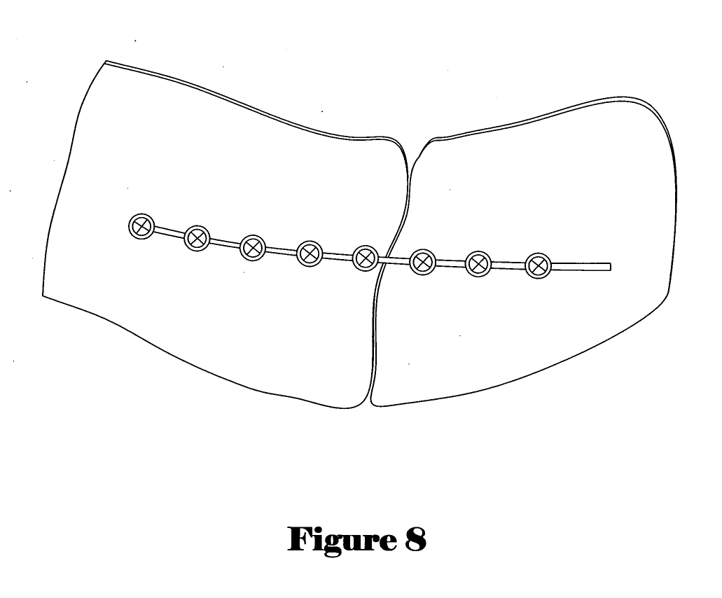

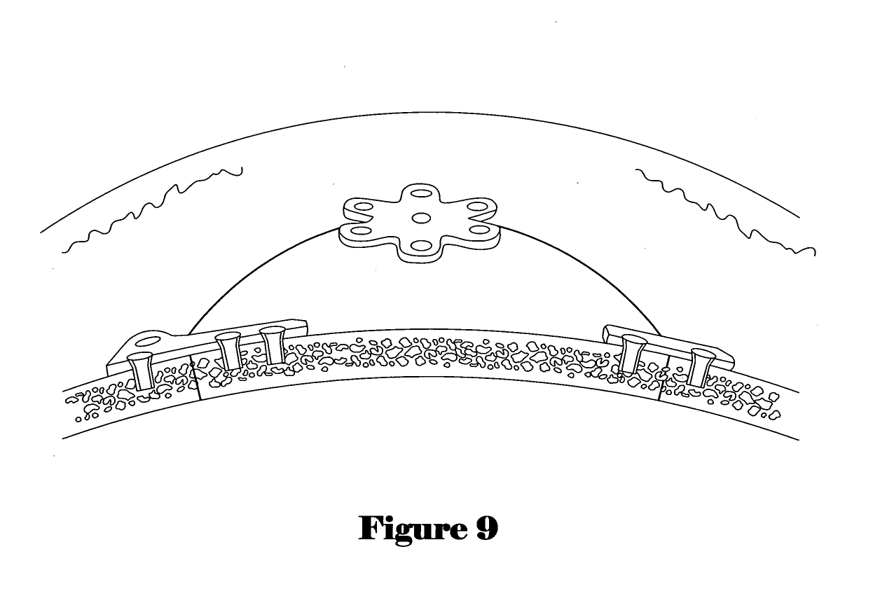

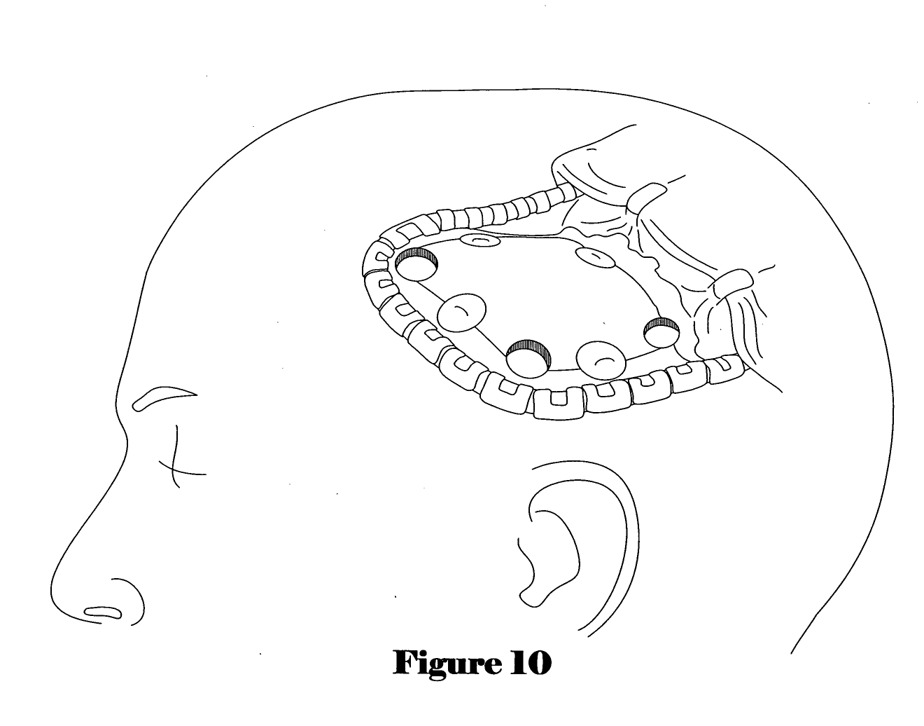

### DETAILED DESCRIPTION OF THE INVENTION

Various embodiments of the present invention are directed to a cranial fixation device for fixing a skull flap to a skull. In one embodiment of the present invention, a cranial fixation device (“fixation device”) is securely fastened inside each perimeter burr hole (“burr hole”) created along a skull/skull-flap border during a craniotomy. The fixation device fixes the skull flap to the skull so that a top surface of the fixation device is flush with an outer surface of the skull and the skull flap. Accordingly, skin may subsequently be re-laid over the site of the craniotomy without visible and/or palpable craniotomy-created depressions and/or protrusions.

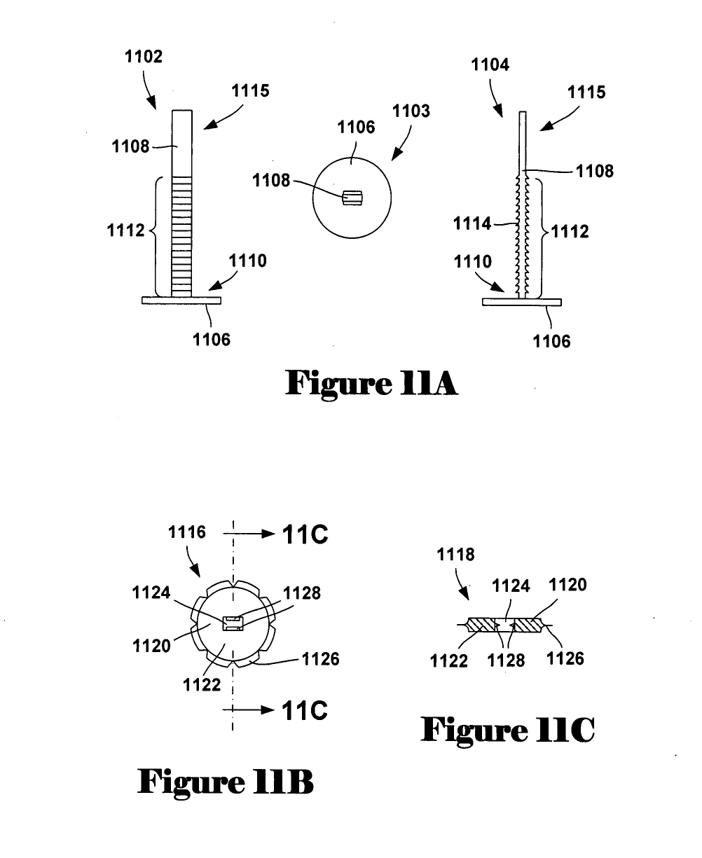

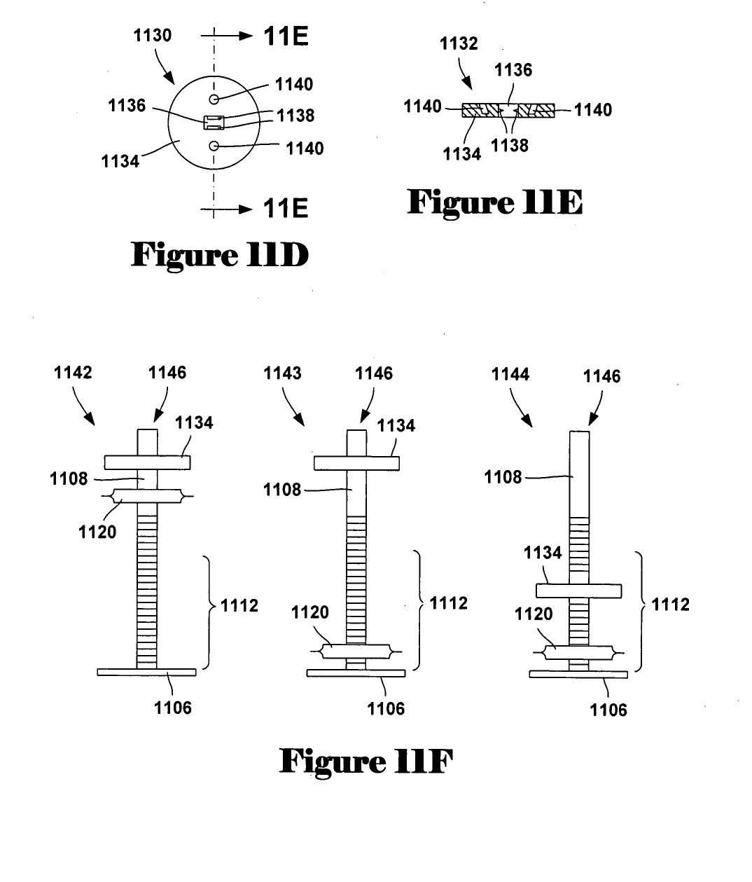

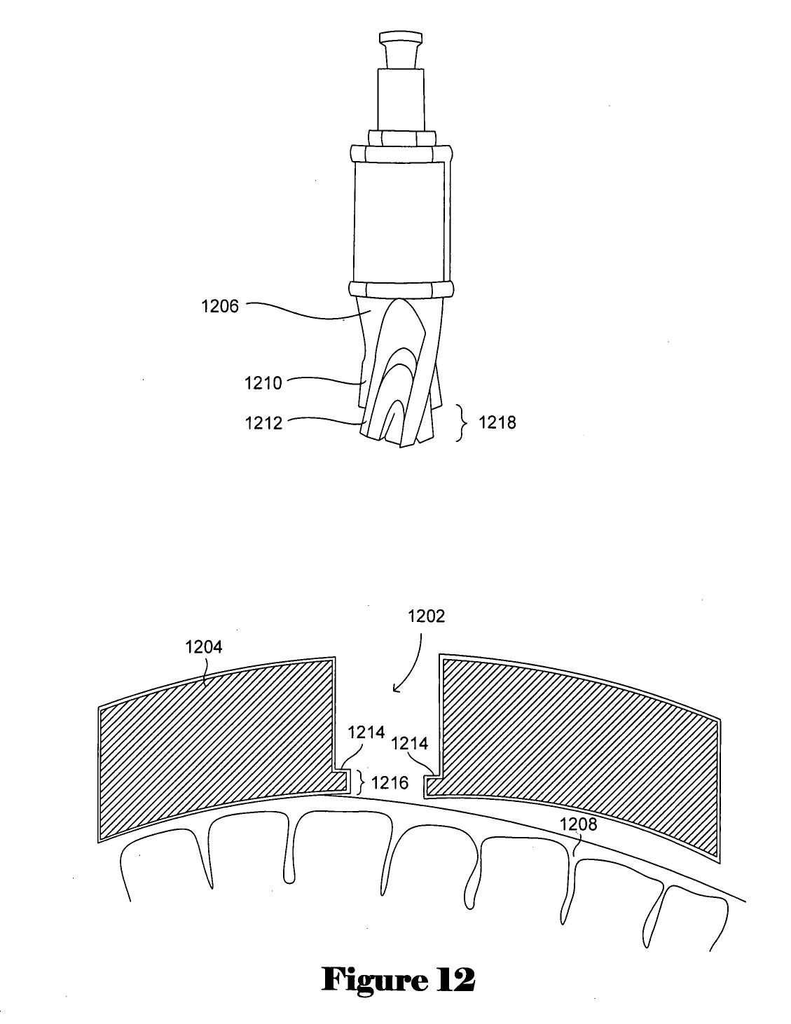

FIG. 11A shows three orthogonal views of a base and stem for a fixation device that represents one embodiment of the present invention. FIG. 11A shows three orthogonal views 1102-1104 of a base 1106 and a stem 1108. The base 1106 is rigid or semi-rigid and roughly disc-shaped with a diameter greater than the diameter of a pre-drilled burr hole. FIG. 11A shows the stem 1108 having a flat, elongated rectangular shape. The stem 1108 attaches to, and is approximately centered on, a top surface of the base 1106 at a proximal end 1110 of the stem 1108 and extends in a direction that is approximately orthogonal to the base 1106. In one embodiment of the present invention, the base 1106 and the stem 1108 are a unitary structure. In alternate embodiments of the present invention, the base 1106 and the stem 1108 are separate components and may be either removably or permanently interconnected subsequent to fabrication. The stem 1108 has a length that is at least as long as the thickest portion of a skull into which the stem 1108 is to be implanted. The stem 1108 contains an integrated gear rack 1112 on opposite sides of the stem 1108 that extends a portion of the length of the stem 1108, beginning in proximity to the proximal end 1110 of the stem 1108. The gear rack 1112 includes a number of teeth, such as tooth 1114. FIG. 11A shows the teeth extending outward in a downward direction. In alternate embodiments of the present invention, the teeth extend outward in a roughly horizontal orientation. The stem 1108 also includes a distal end 1115 of variable length. In one embodiment of the present invention, a distal end of a stem does not contain a gear rack. In another embodiment of the present invention, the stem does not contain a gear rack.

FIGS. 11B and 11C show a top view and a cross-sectional view of a fan washer for a fixation device that represents one embodiment of the present invention. FIG. 11B shows a top view 1116 and FIG. 11C shows a cross-sectional view 1118 of a roughly disc-shaped fan washer 1120. The fan washer 1120 includes a body portion 1122 with a fan-washer interconnection aperture 1124 that is similar in shape to the stem 1108 and a number of firm, yet flexible fan blades extending outward from the periphery of the body portion 1122, such as fan blade 1126. The diameter of the body portion 1122 of the fan washer 1120 is smaller than the diameter of a pre-drilled burr hole. However, the diameter of the fan washer 1120 with the fan blades is greater than the diameter of the pre-drilled burr hole, thus enabling the fan washer 1120 to fit snugly within the pre-drilled burr hole when the fan blades are bent.

The size and shape of the fan-washer interconnection aperture 1124 may be similar to the circumference of the stem 1108 in order to facilitate the movement of the fan washer 1120 along the stem 1108. The fan-washer interconnection aperture includes a fan-washer ratchet 1128. When the stem 1108 is passed through the fan-washer interconnection aperture 1124, the fan washer 1120 may freely slide up and down the length of the stem 1108 so long as the fan-washer interconnection aperture 1124 does not pass over the gear rack 1112. Once the fan-washer interconnection aperture 1124 passes over the gear rack 1112, the fan-washer ratchet 1128 engages the teeth of the gear rack 1112. When the fan-washer ratchet 1128 engages the teeth of the gear rack 1112, the fan washer 1120 may move in a proximal direction towards the base 1106 with a relatively small amount of force being applied to the stem 1108 and/or the fan washer 1120. However, when the fan-washer ratchet 1128 engages the teeth of the gear rack 1112, the fan washer 1120 is prevented from moving in a distal direction away from the base 1106 without a relatively large amount of force being applied to the stem 1108 and/or the fan washer 1120. Note that, in embodiments of the present invention that exclude a gear rack, the fan-washer interconnection aperture may not include a fan-washer ratchet.

FIGS. 11D and 11E show a top view and a cross-sectional view of a cap for a fixation device that represents one embodiment of the present invention. FIG. 11D shows a top view 1130 and FIG. 11E shows a cross-sectional view 1132 of a roughly disc-shaped cap 1134. The cap 1134 includes a cap interconnection aperture 1136 with an internal cap ratchet 1138. The size and shape of the cap interconnection aperture 1136 enables the cap 1134 to slide along the stem 1108 and engage the gear rack 1112 in a similar manner to the fan washer 1120. The cap 1134 is slightly smaller in diameter than a pre-drilled burr hole. In one embodiment of the present invention, a pre-drilled burr hole is roughly 14 mm in diameter, a base is roughly 16 mm in diameter, a stem is roughly 4 mm wide and roughly 50 mm tall, a fan washer is roughly 15 mm in diameter (including the fan blades), and a cap is roughly 12 mm in diameter. Note that, in embodiments of the present invention that exclude a gear rack, the cap interconnection aperture may not include a cap ratchet.

It may occasionally become necessary to remove a fixation device, should a patient need reoperation and skull flap removal. For example, fixation-device removal may be necessary when a patient incurs a post operative hematoma or infection. In one embodiment of the present invention, the cap 1134 may be slid off the stem 1108. To facilitate cap removal, the cap 1134 also includes two slanted removal apertures 1140. The two slanted removal apertures 1140 are sized and shaped so that the ends of a standard towel clamp may be inserted into the two slanted removal apertures 1140 and used to provide leverage for sliding the cap 1134 along the length of the stem 1108.

FIG. 11F shows three side views of a fixation device that represents one embodiment of the present invention. In FIG. 11F, three side views 1142-1144 are shown of a fixation device 1146. In the side views 1142-1144, the stem 1108 has been passed through the fan-washer interconnection aperture 1124 (not shown in FIG. 11F) of the fan washer 1120 and the cap interconnection aperture 1136 (not shown in FIG. 11F) of the cap 1134. In the first side view 1142, neither the fan washer 1120 nor the cap 1134 are engaged by the gear rack 1112. In the second side view 1143, the fan washer 1120 has been slid proximally towards the base 1106 and is engaged by the gear rack 1112. However, the cap 1134 is not engaged by the gear rack 1112. In the third side view 1144, the fan washer 1120 and the cap 1134 are both engaged by the gear rack 1112.

FIG. 12 shows a cross-section of a burr hole drilled into a skull by a drill bit from a cranial perforator. In FIG. 12, a burr hole 1202 is created in a skull 1204 by a spinning drill bit 1206 on a cranial perforator (not shown in FIG. 12). In FIG. 12, the drill bit 1206 has drilled completely through the skull 1204 without damaging the underlying dura 1208, which is shown separated from an inner surface of the skull 1204 to the right of the burr hole 1202. The drill bit 1206 contains two layers of cutting surfaces, a wide upper cutting surface 1210 and a narrow lower cutting surface 1212. Typically, a cranial perforator is designed with a clutch that disengages the drill bit 1206 from a drill shaft and ceases further drilling when the lower cutting surface 1212 has cut completely through the skull 1204 so that neither the dura 1208, nor any other underlying tissues or structures, suffer injury. Because the drill bit 1206 contains two layers of cutting surfaces 1210 and 1212, and the lower cutting surface 1212 is stopped, by the clutch, from penetrating beyond the lower surface of the skull into the dura, a ledge 1214 is created in the burr hole 1202 when the drill bit 1206 is removed from the burr hole 1202. The ledge has a thickness 1216 approximately equal to the length 1218 that the lower cutting surface 1212 extends beyond the tip of the upper cutting surface 1210.

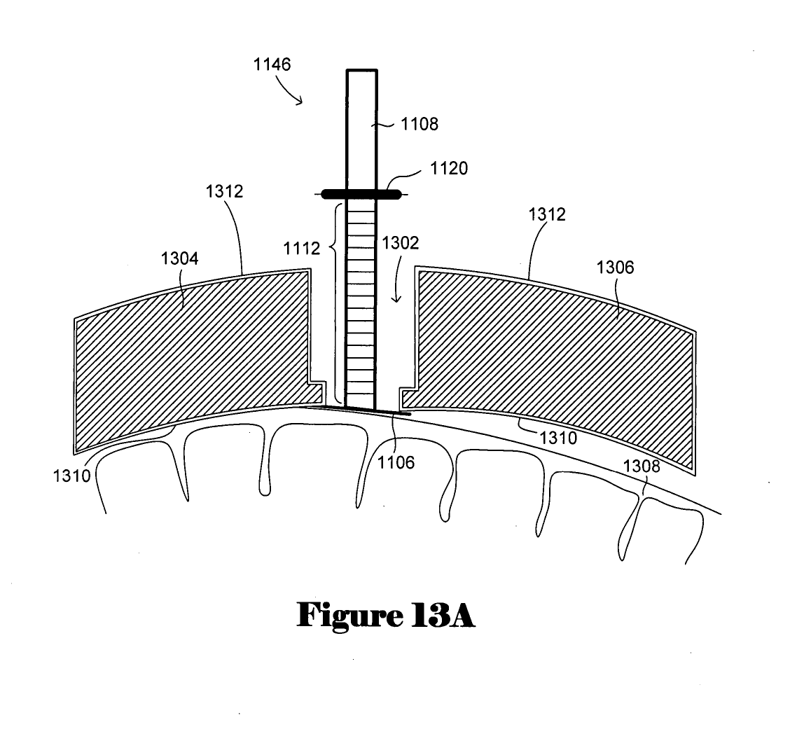

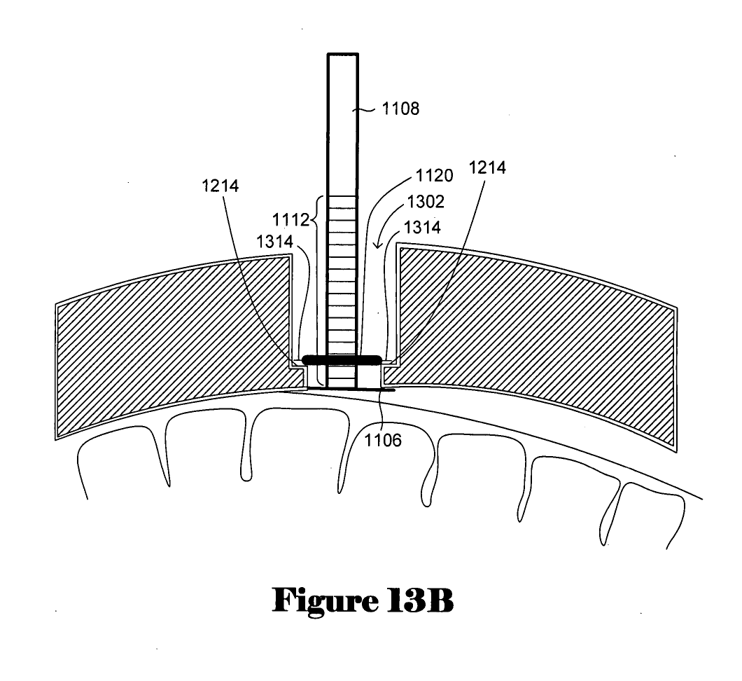

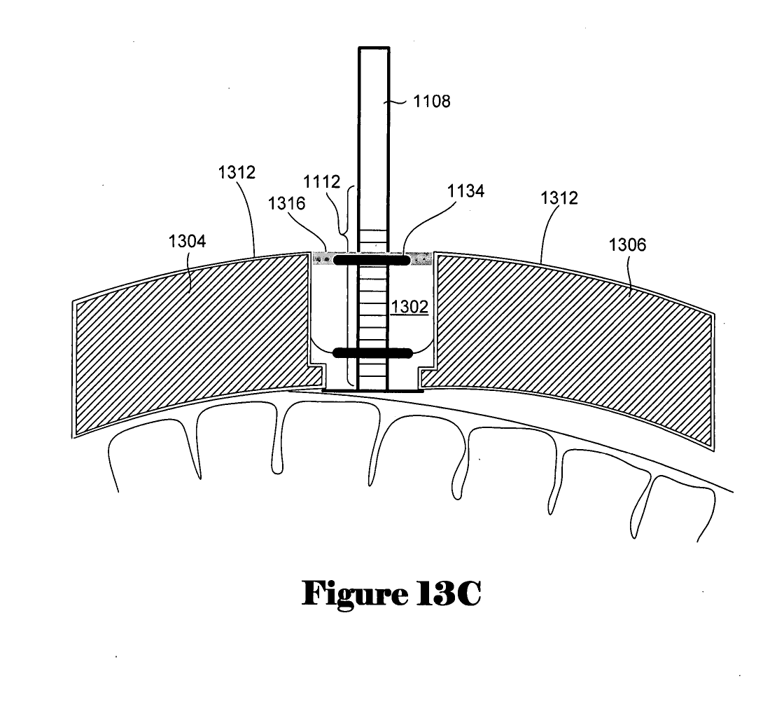

After a craniotomy has been performed, a skull flap is returned to its former position and fixed in place on a skull. FIG. 13A shows a first step for fixing a skull flap to a skull using the fixation device shown in FIGS. 11A-11F that represents one embodiment of the present invention. In FIG. 13A, the fixation device 1146 is shown placed in a burr hole 1302 between a skull 1304 and a skull flap 1306. The base 1106 of the fixation device 1146 is placed between the dura 1308 and an inner surface 1310 of both the skull 1304 and the skull flap 1306, with the stem 1108 extending through the burr hole 1302 beyond an outer surface 1312 of the skull 1304 and the skull flap 1306. The fan washer 1120 is mated with the stem 1108, but is positioned above the gear rack 1112. The base 1106 has a larger diameter than the diameter of a proximal portion of the burr hole 1302, thus allowing the base/stem assembly to hold the skull flap 1306 in position while the fan washer 1120 is being slid down the stem 1108 and into the burr hole 1302. Note that, when a skull flap is separated from a skull, a portion of a burr hole is along the perimeter of a skull and the remaining portion of the burr hole is along the perimeter of a skull flap. In order to position the base beneath an inner surface of both the skull and skull flap, it may be necessary to first position the base underneath the skull portion of the burr hole and subsequently to return the skull flap to its former position with the skull flap portion of the burr hole aligned with the skull portion of the burr hole. Note also that, when a stem is positioned in a burr hole between a skull and skull flap, an associated gear rack extends along the length of the stem to a position on the stem that is beyond the plane of the outer surfaces of the skull and skull flap.

FIG. 13B shows a second step for fixing a skull flap to a skull using the fixation device shown in FIGS. 11A-11F that represents one embodiment of the present invention. In FIG. 13B, the fan washer 1120 has been moved proximally along the stem 1108 towards the base 1106, until the fan washer 1120 is resting on the ledge 1214 created by the drilling process, as discussed above with reference to FIG. 12. Although a number of fan blades 1314 around the periphery of the fan washer 1120 make the diameter of the fan washer 1102 greater than the diameter of the burr hole 1302, the fan blades 1314 are able to bend upward and press against the walls of the burr hole 1302. Consequently, once the fan washer 1120 is slid proximally to the ledge 1214, the fan washer 1120 is locked in place by the bent fan blades 1312 and the ledge 1214 is tightly held in position between the base 1106 and the fan washer 1120.

FIG. 13C shows a third step for fixing a skull flap to a skull using the fixation device shown in FIGS. 11A-11F that represents one embodiment of the present invention. In FIG. 13C, the cap 1134 has been mated with the stem 1108 and slid proximally along the stem 1108 until the cap 1134 is engaged by the gear rack 1112 and a top surface 1316 of the cap 1134 is flush with the outer surfaces 1312 of both the skull 1304 and the skull flap 1306. FIG. 13D shows a fourth step for fixing a skull flap to a skull using the fixation device shown in FIGS. 11A-11F that represents one embodiment of the present invention. In FIG. 13D, the distal end 1115 (not shown in FIG. 13D) of the stem 1108 has been cleaved in proximity to the top surface 1316 of the cap 1134 creating a smooth, secure, and cosmetic surface over which peeled-away skin may be re-laid.

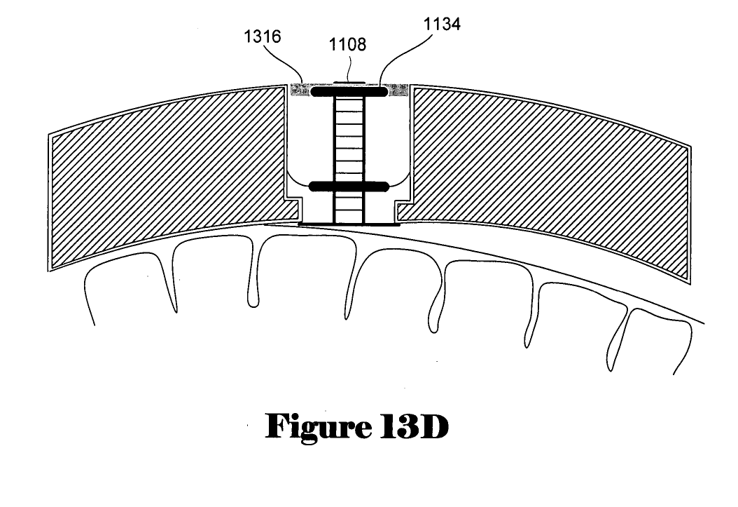

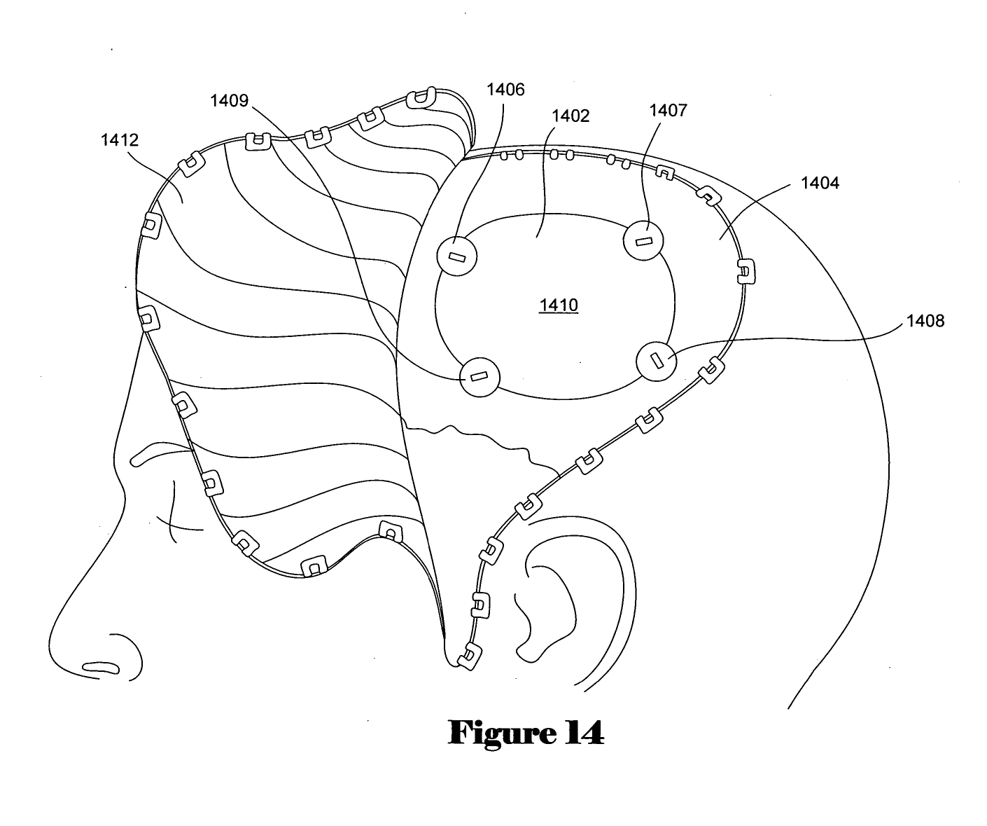

The procedure discussed above, with reference to FIGS. 13A-13D may be repeated for each craniotomy-created burr hole. Once a distal portion of each stem extending above a top surface of each cap is cleaved, the flap of skin and muscle may be replaced and reconnected. FIG. 14 shows a skull flap fixed to a skull by four fixation devices that represent one embodiment of the present invention. A skull flap 1402 is shown fixed to a skull 1404 using four fixation devices 1406-1409. The four fixation devices 1406-1409 extend through burr holes drilled during a craniotomy. Top surfaces of the caps for each of the four fixation devices 1406-1409 are flush with the outer surfaces 1410 of both the skull 1404 and the skull flap 1402 and the distal ends of the stems are cleaved in proximity to the top surfaces of the caps. When the peeled-away skin flap 1412 is replaced over the skull flap 1402 and reconnected to the skin on the remainder of the skull, the underlying fixation device enables the skull flap to be fixed to the skull without creating visible and/or palpable protrusions and/or indentations.

When a patient needs to undergo a subsequent craniotomy in the same region of the skull, once the skin and muscles are peeled away to expose the skull, a medical professional may use a pair of towel clamps to remove the caps. The medical professional may insert the ends of the towel clamp into the two slanted removal apertures on the top surface of each cap and exert the amount of force needed to remove each cap. Although the remaining pieces of the fixation device remain in place, the burr hole is accessible for placement of a craniotome for resawing of the bone flap. Once the skull flap is removed, the remaining pieces of the fixation device may also be removed. Alternately, when reopening a recently-created skull flap, a medical professional may use a pinching tool, such as a pair of pliers, to completely remove the remaining pieces of a fixation device after removing the cap. The medical professional may grasp the stem and exert enough force to pull the base, fan washer, and stem of the fixation device out through the burr hole.

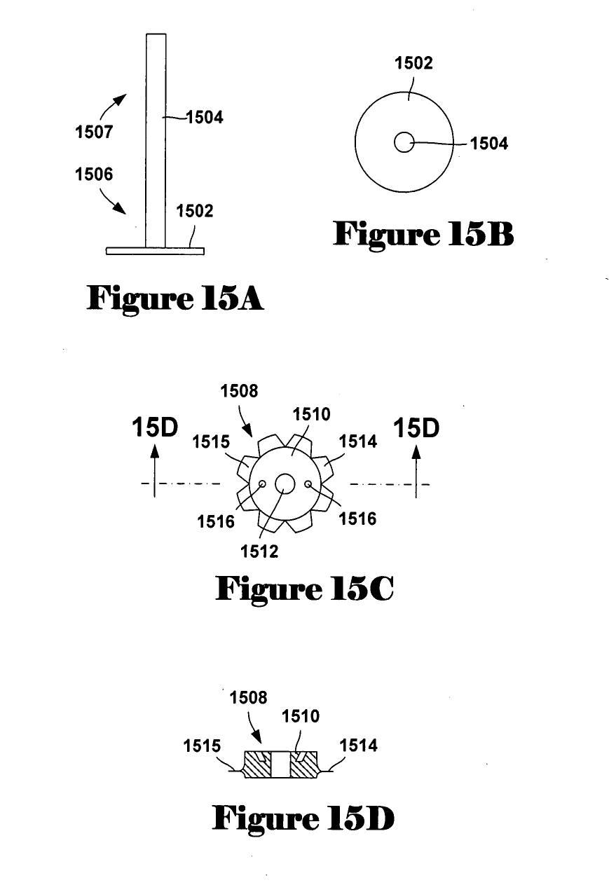

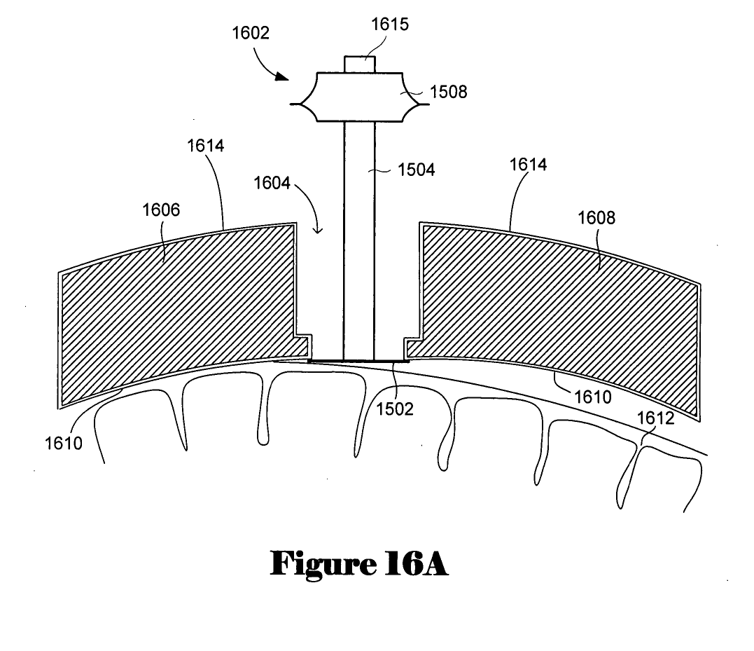

In alternate embodiments of the present invention, a fixation device may be fabricated without a cap and/or a gear rack. FIG. 15A shows a side view of a base and an interconnected stem for a fixation device that represents one embodiment of the present invention. FIG. 15A shows a base 1502 and a stem 1504. The stem 1504 attaches to the base 1502 at a proximal end 1506 of the stem 1504 and extends in a direction that is approximately orthogonal to the base 1502 to a distal end 1507. The stem 1504 has a length that is at least as long as the thickest portion of the skull into which the stem 1504 is to be implanted. FIG. 15B shows a top view of a base and an interconnected stem for a fixation device that represents one embodiment of the present invention. In FIG. 15B, the stem 1504 has a round circumference and extends from roughly the center of a top surface of the base 1502.

FIG. 15C shows a top view of a fan washer for a fixation device that represents one embodiment of the present invention. A fan washer 1508 includes a body portion 1510 with a fan-washer interconnection aperture 1512 and a number of firm, yet flexible fan blades extending outward from the periphery of the body portion 1510, such as fan blades 1514 and 1515. The fan washer 1508 also includes two slanted removal apertures 1516 on the body portion 1510 flanking the fan-washer interconnection aperture 1512. Note that the diameter of the body portion 1510 of the fan washer 1508 is slightly smaller than the diameter of a pre-drilled burr hole. However, the diameter of the fan washer 1508 with the fan blades is larger than the diameter of a pre-drilled burr hole, thus enabling the fan washer 1508 to fit snugly within the pre-drilled burr hole when the fan blades are bent. The size and shape of the fan-washer interconnection aperture 1512 may be similar to the circumference of the stem 1504 in order to facilitate the movement of the fan washer 1508 along the stem 1504.

In alternate embodiments of the present invention, the fan washer includes a fan-washer ratchet in the fan-washer interconnection aperture that mates with a gear rack extending along a portion of the length of a stem. When the stem is passed through the fan-washer interconnection aperture, the fan washer may freely slide up and down the length of the stem so long as the fan-washer interconnection aperture does not pass over the gear rack. Once the fan-washer interconnection aperture passes over the gear rack, the fan-washer ratchet engages the teeth of the gear rack. When the fan-washer ratchet engages the teeth of the gear rack, the fan washer may move in a proximal direction towards the base with a relatively small amount of force being applied to the stem and/or the fan washer. However, when the fan-washer ratchet engages the teeth of the gear rack, the fan washer is prevented from moving in a distal direction away from the base without a relatively large amount of force being applied to the stem and/or the fan washer. FIG. 15D shows a cross-sectional view of a fan washer for a fixation device that represents one embodiment of the present invention. The fan blades 1514 and 1515 are thinner than the body portion 1510 of the fan washer 1508 and are designed to be firm, yet flexible. In FIG. 15D, the fan blades 1514 and 1515 are shown towards the bottom of the sides of the body portion 1510 of the fan washer 1508.

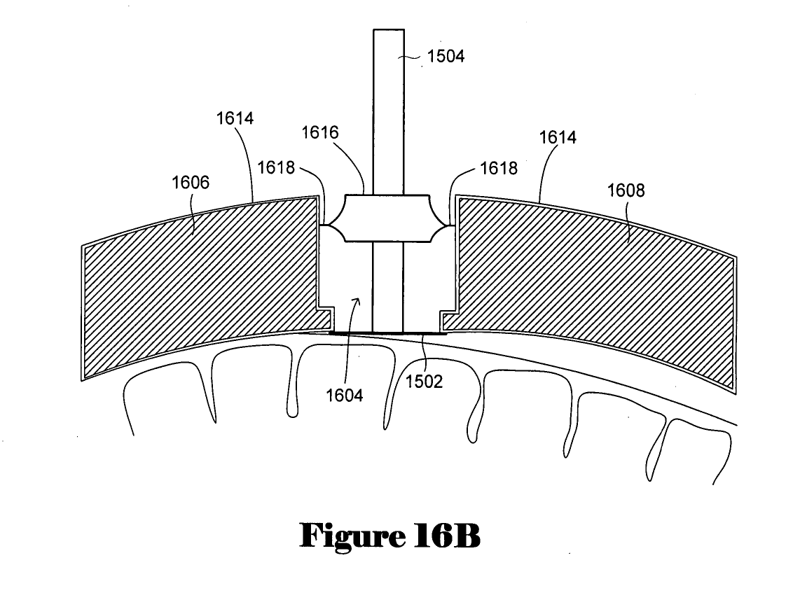

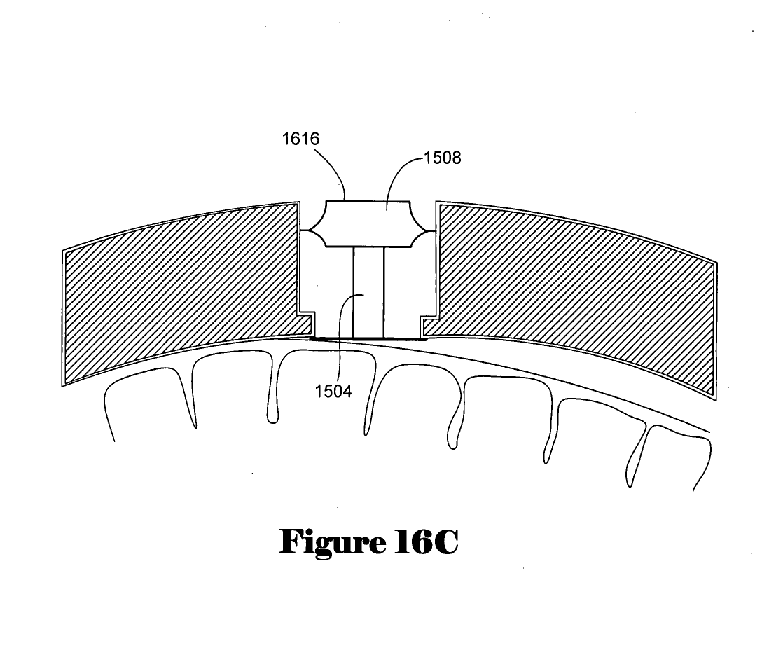

FIG. 16A shows a first step for fixing a skull flap to a skull using the fixation device shown in FIGS. 15A-15D that represents one embodiment of the present invention. In FIG. 16A, a fixation device 1602 with a base 1502, a stem 1504, and a fan washer 1508 is shown placed in a burr hole 1604 between a skull 1606 and a skull flap 1608. The base 1502 is placed beneath inner surfaces 1610 of both the skull 1606 and the skull flap 1608, and above a section of stripped-away dura 1612. The stem 1504 extends outward through the burr hole 1604, so that a distal end 1614 of the stem 1504 is positioned beyond outer surfaces 1614 of the skull 1606 and the skull flap 1608. The fan washer 1508 is mated with the stem 1504 in proximity to the distal end 1614 of the stem 1504. Note that the base 1502 has a larger diameter than the diameter of a proximal portion of the burr hole 1604 to anchor the fixation device 1602 in place so that the fixation device 1602 cannot be lifted up through the burr hole 1604 without applying a large amount of force.

FIG. 16B shows a second step for fixing a skull flap to a skull using the fixation device shown in FIGS. 15A-15D that represents one embodiment of the present invention. In FIG. 16B, the fan washer 1508 has been pushed proximally along the stem 1504 towards the base 1502 so that a top surface 1616 of the fan washer 1508 is flush with the outer surfaces 1614 of both the skull 1606 and the skull flap 1608. Note that the fan blades 1618 around the periphery of the fan washer 1508 make the diameter of the fan washer 1508 larger than the diameter of the burr hole 1604. Consequently, the fan blades 1618 bend upward and press against the walls of the burr hole 1604 when the fan washer 1508 is pressed into the burr hole 1604. Once the fan washer 1508 is pushed proximally until the top surface 1616 of the fan washer 1508 is flush with the outer surfaces 1614 of both the skull 1606 and the skull flap 1608, the fan washer 1508 is held in place by the bent fan blades 1618. Note that, when a fan washer includes a ratchet in a fan-washer interconnection aperture and a stem includes a gear rack, the fan washer may further be held in place by the internal ratchet engaging the gear rack.

FIG. 16C shows a third step for fixing a skull flap to a skull using the fixation device shown in FIGS. 15A-15D that represents one embodiment of the present invention. In FIG. 16C, the distal end 1614 (not shown in FIG. 16C) of the stem 1504 has been cleaved in proximity to the top surface 1616 of the fan washer 1508, creating a smooth, secure, and cosmetic surface over which peeled-away skin may be re-laid. The procedure discussed above, with reference to FIGS. 16A-16C may be repeated for each burr hole created during the removal of a skull flap during a craniotomy. Once the distal end of each stem is cleaved in proximity to the top surface of each fan-washer cap, the skin flap may be replaced and reconnected, as discussed above with reference to FIG. 14. Note that, in the procedure described with reference to FIGS. 16A-16C a ledge formed during the drilling of a burr hole is not sandwiched between a base and a fan washer because the fan washer remains in a more distal position within the burr hole so that the top surface of the fan washer is flush with the outer surfaces of the skull and skull flap.

When a patient needs to undergo a subsequent craniotomy in the same region of the skull, once the skin and muscles are peeled away to expose the skull, a medical professional may use a pair of towel clamps to remove the fan washers. The medical professional may insert the ends of the towel clamp into the two slanted removal apertures on the top surface of each fan washer and exert the amount of force needed to remove each fan washer. Although the remaining pieces of the fixation device remain in place, the burr hole is accessible for placement of a craniotome for resawing of the bone flap. Once the skull flap is removed, the remaining pieces of the fixation device may also be removed. Alternately, when reopening a recently-created skull flap, a medical professional may use a pinching tool, such as a pair of pliers, to completely remove a fixation device after removing the fan washer. The medical professional may grasp the stem and exert enough force to pull the base and the stem of the fixation device out through the burr hole.

Craniotomies may, on occasion, be performed where one or more access burr holes are drilled, in addition to the burr holes drilled along the perimeter of a skull flap. Access burr holes may be drilled in order to provide access to the epidural space beneath the portion of the skull to be subsequently removed as a skull flap. Access burr holes may be drilled, for example, during a dural-based tumor removal or when the dura is strongly adherent to an inner surface of the skull. When access burr holes are created in a skull, fan washers may be used to cover the tops of the access burr holes. The fan washers may be used to create a smooth surface over top of the access burr holes without the need of holding a skull flap flush with a skull. Accordingly, fan washers may be placed in access burr holes without the need for placing a corresponding stem and/or base, since the placement of the fan washers is for cosmesis and is not used to fix a skull flap to a skull.

A base, a stem, a fan washer, and a cap may be fabricated from any of a number of tough, flexible or non-flexible materials suitable for implantation in the skull. Note that, because of the relative thinness of the fan blades, when non-flexible materials are used to fabricate a fan washer, the fan blades may still be firm, yet flexible. In alternate embodiments of the present invention, a base, stem, fan washer, and cap are manufactured from biodegradable materials that may function as a scaffolding to promote bone growth as the material biodegrades. The materials used to manufacture a base, stem, fan washer, and cap may also be impregnated with various substances, such as anti-bacterial agents and antibiotic. Various different materials may be used for each of the individual pieces. For example, a base may be fabricated from a non-flexible material impregnated with an antibiotic, while the stem is fabricated from a biodegradable material and the cap is fabricated from a flexible material.

Additional modifications within the spirit of the invention will be apparent to those skilled in the art. For example, the sizes and shapes of burr holes drilled into a skull may vary. Consequently, the sizes and shapes of a base, stem, fan washer, and cap may vary as well. In alternate embodiments of the present invention, a base may be rectangular-shaped, oblong-shaped, or other suitable shape. Similarly, a stem may be rectangular-shaped, tubular-shaped, triangular-shaped, oblong-shaped, or other suitable shape. A stem may extend from a location on a base other than roughly the center of a top surface. In embodiments of the present invention where a stem includes a gear rack, the gear rack may or may not extend to the distal end of a stem. A base and a stem may be either a unitary structure or separate pieces that may be either permanently or removably interconnected to each other subsequent to fabrication. When a base and a stem are removably attached to one another, the attachment may be formed in any of a number of different ways, such as snapping, screwing, or pressing the proximal end of a stem into a receiving aperture in a base. When a burr hole is a shape other than round, the shape of a fan washer and cap may need to be changed to accommodate the space created by the burr hole. The number of fan washers around the periphery of a fan washer may be varied as well. Additionally, fan blades may vary in rigidity and width to vary how tightly a fan washer is held in place. Moreover, fan blades may be placed at various locations along the periphery of fan washer. For example, one or more fan blades may extend from near a top region of the side of a body portion of a fan washer, while one or more fan blades may also extend from a bottom region of the side of a body portion the fan washer. Fixation devices may be used to fix pieces of skull together that were not removed during a craniotomy. For example, a fixation device may be used to fix together two or more pieces of skull separated by a crack in the skull caused by a traumatic impact. In addition to fixation devices, bone fragments and/or other suitable filling materials may also be inserted into burr holes.

The foregoing detailed description, for purposes of illustration, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Obviously many modifications and variation are possible in view of the above teachings. The embodiments were chosen and described in order to best explain the principles of the invention and its practical applications and to thereby enable others skilled in the art to best utilize the invention and various embodiments with various modifications as are suited to the particular use contemplated.

1. A cranial fixation device for fixing a skull flap to a skull underlain with dura mater, the cranial fixation device comprising:
a base for positioning beneath a burr hole drilled at a boundary between the skull and skull flap and above the underlain dura mater; and
a stem with a proximal end and a distal end, the proximal end interconnected to the base and the distal end extending through the burr hole; and
a fan washer with a fan-washer interconnection aperture and a number of peripheral fan blades, the fan-washer interconnection aperture for interconnecting the fan washer to the stem, and the number of peripheral fan blades for anchoring the fan washer to a burr-hole wall.

2. The cranial fixation device of claim 1 wherein the stem includes a gear rack, the gear rack containing a number of teeth.

3. The cranial fixation device of claim 2 wherein the fan-washer interconnection aperture includes a fan-washer ratchet for engaging one or more teeth on the gear rack.

4. The cranial fixation device of claim 2 further including a cap with a cap interconnection aperture, the cap interconnection aperture for interconnecting the cap to the stem.

5. The cranial fixation device of claim 4 wherein the cap interconnection aperture includes a fan-washer ratchet for engaging one or more teeth on a gear rack.

6. The cranial fixation device of claim 4 wherein the cap further includes a top surface, the top surface including a number of slanted removal apertures shaped to mate with towel clamps.

7. The cranial fixation device of claim 1 wherein the fan washer further includes a top surface, the top surface including a number of slanted removal apertures shaped to mate with towel clamps.

8. The cranial fixation device of claim 1 wherein the fan washer has a diameter that is greater than the diameter of the burr hole.

9. The cranial fixation device of claim 1 wherein the base is fabricated from a rigid material and has a diameter that is greater than the diameter of a proximal portion of the burr hole.

10. The cranial fixation device of claim 1 wherein the peripheral fan blades are fabricated from a flexible material, the peripheral fan blades anchoring the fan washer to the burr-hole wall by pressing against the burr-hole wall and bending when the fan washer is interconnected to the stem and inserted into the burr hole.

11. The cranial fixation device of claim 1 wherein the cranial fixation device is fabricated from one or more biodegradable substances.

12. The cranial fixation device of claim 1 wherein the cranial fixation device is impregnated with one or more of
antibacterial agents; and
antibiotic.

13. A fixed cranial structure comprising:
a skull underlain with dura mater, the skull having a removable skull flap and a burr hole along a boundary between the skull and the skull flap; and
a cranial fixation device within and beneath the burr hole, the cranial fixation device including a base positioned beneath the burr hole and above the dura mater, a stem with a proximal end and a distal end, the proximal end interconnected to the base and the distal end extending through the burr hole, and a fan washer with a fan-washer interconnection aperture and a number of peripheral fan blades, the fan-washer interconnection aperture for interconnecting the fan washer to the stem, the number of peripheral fan blades for anchoring the fan washer to a wall in the burr hole.

14. The fixed cranial structure of claim 13 wherein the stem includes a gear rack, the gear rack containing a number of teeth.

15. The fixed cranial structure of claim 14 wherein the fan-washer interconnection aperture includes a fan-washer ratchet for engaging one or more teeth on the gear rack.

16. The fixed cranial structure of claim 14 further including a cap with a cap interconnection aperture, the cap interconnection aperture for interconnecting the cap to the stem.

17. The fixed cranial structure of claim 16 wherein the cap interconnection aperture includes a fan-washer ratchet for engaging one or more teeth on a gear rack.

18. The fixed cranial structure of claim 16 wherein the cap further includes a top surface, the top surface including a number of slanted removal apertures shaped to mate with towel clamps.

19. The fixed cranial structure of claim 13 wherein the fan washer further includes a top surface, the top surface including a number of slanted removal apertures shaped to mate with towel clamps.

20. The fixed cranial structure of claim 13 wherein the peripheral fan blades are fabricated from a flexible material, the peripheral fan blades anchoring the fan washer to the burr-hole wall by pressing against the burr-hole wall and bending when the fan washer is interconnected to the stem and inserted into the burr hole.

21. A method for fixing a skull underlain with dura mater to a removable skull flap with a burr hole along a boundary between the skull and the skull flap, the method comprising:
providing a cranial fixation device, the cranial fixation device including a base, an interconnected stem with a proximal end and a distal end, and an interconnectable fan washer with a fan-washer interconnection aperture and a number of peripheral fan blades;
positioning the base beneath the burr hole and above the dura mater;
mating the fan-washer interconnection aperture with the distal end of the stem;
sliding the fan washer proximally down the stem into the burr hole; and
cleaving the distal end of the stem.

22. The method of claim 21 wherein the stem includes a gear rack, the gear rack containing a number of teeth.

23. The method of claim 22 wherein the fan-washer interconnection aperture includes a fan-washer ratchet for engaging one or more teeth on the gear rack.

24. The method of claim 22 further including a cap with a cap interconnection aperture, the cap interconnection aperture for interconnecting the cap to the stem and sliding the cap proximally down the stem into the burr hole.

25. The method of claim 24 wherein the cap interconnection aperture includes a fan-washer ratchet for engaging one or more teeth on a gear rack.

26. The method of claim 24 wherein the cap further includes a top surface, the top surface including a number of slanted removal apertures shaped to mate with towel clamps.

27. The method of claim 21 wherein the fan washer further includes a top surface, the top surface including a number of slanted removal apertures shaped to mate with towel clamps.

28. The method of claim 21 wherein the peripheral fan blades are fabricated from a flexible material, the peripheral fan blades anchoring the fan washer to the burr-hole wall by pressing against the burr-hole wall and bending when the fan washer is interconnected to the stem and inserted into the burr hole.
